(19) United States
(12) Patent Application Publication
(10) Pub. No.: US 2008/0109018 A1
(43) Pub. Date: May 8, 2008

(54) SURGICAL CLIP

(75) Inventor: Jean-Jacques MARTIN, Bourg-En-Bresse (FR)

Correspondence Address: MORGAN, LEWIS & BOCKIUS LLP, 1701 MARKET STREET, PHILADELPHIA, PA, 19103-2921, US

(73) Assignee: SMALL BONE INNOVATIONS INTERNATIONAL SAS, Peronnas (FR)

(21) Appl. No.: 11/854,260

(22) Filed: Sep. 12, 2007

(30) Foreign Application Priority Data
Sep. 12, 2006 (FR) 0607949

(51) Int. Cl. A61B 17/08; A61B 17/56

(52) U.S. Cl. 606/151; 606/73

(57) ABSTRACT

This surgical clip (1), comprises, on at least one lateral arm (3), several successive projecting portions (4), particularly of a tapered shape, forming series of shoulders (5) suitable for opposing, after insertion, the extraction of said lateral arm (3) from the bone.

According to the invention, at least one projecting portion (4) of said lateral arm (3) comprises at least one groove (6) opening onto the shoulder (5) formed by said portion (4), said groove (6) and said shoulder (5) thus delimiting a pointed and sharp material zone (7), suitable for hollowing the bone when, after the intermediate arm (2) of the clip has been cut, said lateral arm (3) is driven in rotation.

### CROSS-REFERENCE TO RELATED APPLICATIONS

This application claims priority to French Patent Application No. 0607949 filed Sep. 12, 2006, currently pending, which is incorporated herein by reference in its entirety.

The present invention relates to a surgical clip. This clip may particularly be used to hold two parts of a bone together or to enable the reinsertion of a ligament on a bone.

It is common to use smooth surgical clips, i.e. formed from a doubly folded wire in order to form one intermediate arm and two parallel side arms, substantially perpendicular to the intermediate arm, intended to be inserted into a bone. The free ends of the side arms are bevelled so as to facilitate their penetration into cortical bone.

The drawback of these conventional clips is that they do not have a very good hold in the bone and therefore are liable to be expelled therefrom under the effect of repeated stress to which they are liable to be subjected.

It is also known to provide attachment lugs on the lateral arms of the clip, particularly in the form of successive tapered portions forming series of shoulders.

These clips offer the advantage of good adhesion in the bone, in the case of both spongy bone and cortical bone. However, these clips have the drawback of being particularly difficult to remove, involving the application of a very strong tensile force and frequently resulting in more or less extensive extraction of the bone areas in which the lateral arms are inserted.

The present invention is intended to remedy this essential drawback, by providing a surgical clip with a very good adhesion potential in bone while being relatively easy to remove, if applicable.

The clip in question comprises, in a manner known per se, on at least one lateral arm, several successive projecting portions, particularly of a tapered shape, forming series of shoulders suitable for opposing, after insertion, the extraction of said lateral arm from the bone. The clip according to the present invention may be comprised of any biocompatible material, including but not limited to, stainless steel, nitinol, or titanium.

According to the invention, at least one projecting portion of said lateral arm comprises at least one groove opening onto the shoulder formed by said portion, said groove and said shoulder thus delimiting a pointed and sharp material zone, suitable for hollowing the bone when, after the intermediate arm of the clip has been cut, said lateral arm is driven in rotation.

The shoulders formed by said successive projecting portions enable perfect anchoring of said arm of the clip, the latter being capable of perfectly withstanding the various types of stress to which it is liable to be subjected.

If a clip needs to be removed, the intermediate arm of the clip is cut, and the fragment(s) of the clip comprising the lateral arm(s) according to the invention are driven in rotation along the axis of each lateral arm, in the direction of the grooves corresponding to an “unscrewing” of this fragment. During this unscrewing, each pointed and sharp portion produces a hollow in the bone that is comparable to a “bore”, which makes it possible to provide a widened passage for each shoulder; consequently, this widened passage make it possible to remove said clip fragment easily, without any substantial extraction of the bone area receiving said lateral arm.

This hollowing of the bone occurs especially as said pointed and sharp portion may be more or less deformed during the impaction of the clip following friction against the bone, said deformation tending to raise the portion slightly and thus favouring the boring effect of the bone during the “unscrewing” of the clip fragment.

Preferentially, each of said successive projecting portions displays a tapered shape.

Due to this tapered shape, the groove displays a comparatively greater depth on the side of the shoulder wherein it opens, and therefore said pointed and sharp portion displays a correspondingly wider bone lead-in area. This width favours the efficacy of the hollowing of the bone produced by said pointed and sharp portion during said unscrewing.

Each groove preferentially displays a triangular cross-section.

Each groove is preferentially oriented in a direction forming an angle of the order of 75 degrees with the plane of the shoulder wherein it opens.

Preferentially, each groove extends such that the direction of unscrewing of each fragment of the clip after cutting of the intermediate arm is the anti-clockwise direction.

The invention will be understood clearly and other characteristics and advantages thereof will emerge with reference to the appended schematic figure, representing, as a non-limitative example, a preferred embodiment of the surgical clip concerned.

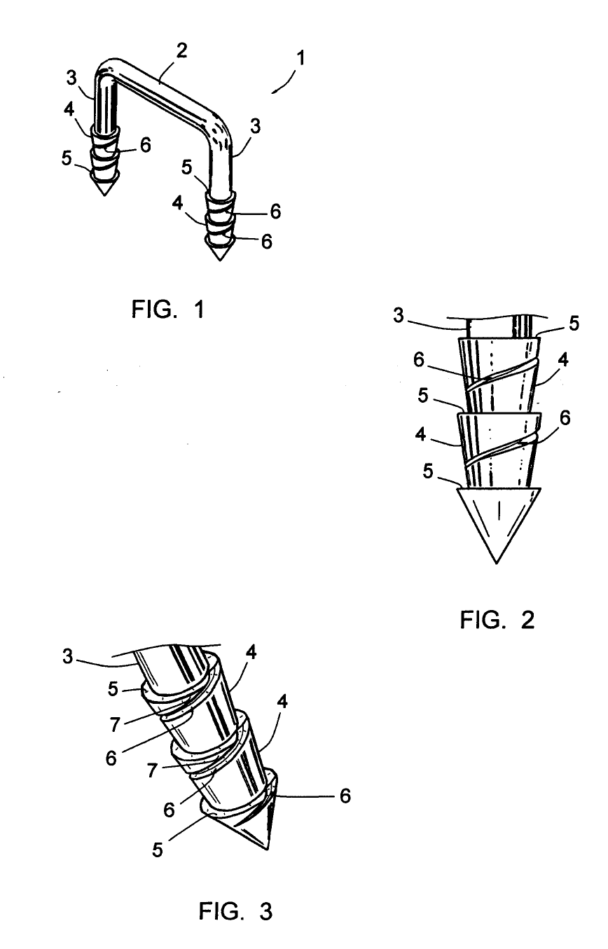

FIG. 1 is a perspective view thereof;

FIG. 2 is a side view of the distal portion of one of the parallel lateral arms comprised by this clip, and

FIG. 3 is a perspective view of this distal portion, from another angle.

FIG. 1 represents a surgical clip 1 comprising an intermediate arm 2 and two parallel lateral arms 3, substantially perpendicular to the arm 2, intended to be inserted into a bone. This clip 1 may particularly be used to hold two parts of a bone together or to enable the reinsertion of a ligament on a bone.

Each lateral arm 3 comprises, on its distal zone, several successive projecting portions 4 of tapered shape, forming series of shoulders 5 suitable for opposing, after insertion, the extraction of said lateral arm 3 from the bone.

As illustrated, each projecting portion 4 comprises a groove 6 opening onto the shoulder 5 formed by said portion. FIG. 3 illustrates more specifically that said groove 6 and said shoulder 5 delimit a pointed and sharp material zone 7 together.

The groove 6 extends in the anti-clockwise direction from the base of a portion 4 to the shoulder 5, has a triangular cross-section, and is oriented in a direction forming an angle of the order of 75 degrees with the plane of the shoulder 5 in which it opens.

In practice, after drilling preliminary holes to receive the arms 3 in the bone, the clip 1 is impacted into the bone area in question. During this impaction, said pointed and sharp portion 7 may be more or less deformed following friction against the bone, this deformation tending to raise it slightly. Once the clip 1 is in place, the shoulders 4 enable strong anchoring of the clip. Said clip is then perfectly capable of withstanding the various types of stress to which it is liable to be subjected.

If the clip needs to be removed, the intermediate arm 2 is cut, and the fragments of the clip comprising the arms 3 are driven in rotation along their axes, in the anti-clockwise direction of each arm 3, corresponding to an “unscrewing” of this fragment. During this unscrewing, each pointed and sharp portion 7 produces a hollow in the bone that is comparable to a “bore”, especially as this portion 7 has been more or less deformed during the impaction of the clip 1 and it displays a relatively wide bone lead-in area due to the fact that it opens into the widest part of the tapered portion 4.

Said “bore” provides a widened passage for each shoulder 5, which makes it possible to be able to remove the clip fragment easily, without substantial extraction of the bone area receiving the lateral arm 3.

As it emerges from the above, the invention provides a surgical clip offering the decisive advantage of very good adhesion potential in the bone while being relatively easy to remove, if applicable.

1. A surgical clip comprising:
at least on lateral arm for insertion into bone comprising several successive projecting portions forming a series of shoulders suitable for opposing the extraction of said at least one lateral arm from bone, wherein at least one said projecting portions comprises at least one groove opening onto at least one of the shoulders, said at least one groove and said at least one shoulder delimiting a pointed and sharp material zone suitable for hollowing the bone when, after the intermediate arm of the clip has been cut, said at least one lateral arm is rotated.

2. A surgical clip according to claim 1, wherein each of said successive projecting portions displays a tapered shape.

3. A surgical clip according to claim 1 wherein said at least one groove has a triangular cross-section.

4. A surgical clip according to claim 1, wherein said at least one groove is oriented in a direction forming an angle of about 75 degrees with the plane of the shoulder in which it opens.

5. A surgical clip according to claim 1, wherein said at least one groove extends such that the direction of unscrewing of each fragment of the clip after the cutting the intermediate arm is the anti-clockwise direction.
